(19) United States
(12) Patent Application Publication
(10) Pub. No.: US 2015/0011014 A1
(43) Pub. Date: Jan. 8, 2015

(54) Method Of Manufacturing And Applications Of Biofunctionalized Amorphous Metal Colloidal Suspensions

(71) Applicants: Kevin Hagedorn, Ann Arbor, MI (US); Andrius Marcinkevicus, Saline, MI (US)

(72) Inventors: Kevin Hagedorn, Ann Arbor, MI (US); Andrius Marcinkevicus, Saline, MI (US)

(21) Appl. No.: 14/323,125

(22) Filed: Jul. 3, 2014

Related U.S. Application Data
(60) Provisional application No. 61/842,417, filed on Jul. 3, 2013.

(51) Int. Cl. G01N 33/543; H01F 41/00; H01F 1/01

(52) U.S. Cl. CPC G01N 33/5434; H01F 1/01; H01F 41/005; G01N 2446/86
USPC 436/501; 252/62.51 R; 252/62.55

(57) ABSTRACT

Disclosed is a process for enhancing the sensitivity of magnetic detection of molecules of interest. The process comprises creating amorphous magnetic metal nanoparticles from a bulk target material comprising at least one magnetic transition metal selected from the group consisting of Ni, Co, and Fe and at least one glass former selected from the group consisting of P, B and Si through the use of a pulsed laser ablation method. The produced amorphous magnetic metal nanoparticles have a large magnetic moment and a large magnetic permeability especially compared to crystalline nanoparticles. One use of the present nanoparticles is in a magnetic immunoassay method.

### RELATED APPLICATIONS

This application claims the benefit of U.S. Provisional Application No. 61/842,417 filed on Jul. 3, 2013.

### STATEMENT REGARDING FEDERALLY SPONSORED RESEARCH

None.

### TECHNICAL FIELD

The present invention relates to the production of amorphous magnetic metal nanoparticle colloids by pulsed laser ablation in a liquid and to the subsequent use of these magnetic nanoparticles in magnetic immunoassay methods.

Immunoassays are based on detection of the binding of an antibody to an antigen. The antigen is also known as the analyte in the assay. The most common detection methods are based on associated enzyme reactions as in the well-known enzyme-linked immunosorbent assay (ELISA) assay methods, detection of radioisotopes that are bound to the antibody, or detection of fluorescent moieties bound to the antibody. A magnetic immunoassay method detects the binding of an antibody to its corresponding antigen by detecting a superparamagnetic nanoparticle conjugated to one element of the pair, either the antigen or the antibody. To be effective in these assays the superparamagnetic nanoparticle needs a high magnetic permeability because an AC magnetic field is needed for signal processing to detect the tiny magnetic moment of the magnetic nanoparticle. A giant magnetoresistance (GMR) sensor can detect tiny magnetic moments by measuring a resistance change in an oscillating magnetic field. To be most useful in these detection methods the superparamagnetic nanoparticle tethered to the GMR sensor surface, by the biomolecules, needs both a large magnetic moment and a large magnetic permeability, two properties which are both size dependent and negatively correlated in crystalline magnetic materials. The magnetic moment of a particle depends almost exclusively on the identity and concentration of magnetic elements in the particle and can be roughly determined using the Slater-Pauling curve. Egami, T. Magnetic amorphous alloys: physics and technological applications. Rep. Prog. Phys. 47, 1601 (1984). Magnetic permeability, in contrast, decreases as the superparamagnetic nanoparticles become larger because the energy barrier between the parallel and anti-parallel orientations increases. This leads to an optimal nanoparticle size for crystalline nanoparticles which is a compromise of magnetic moment and magnetic permeability. Generally, the crystalline nanoparticles of cobalt, for example, must have a size of from 3 to 10 nanometers to exhibit superparamagnetism. Amorphous metals, however, can maintain a high magnetic permeability at sizes larger than the superparamagnetic limit of crystalline nanoparticles, making the particles easier to detect on the surface of a GMR sensor. Thus, it would be highly desirable to create amorphous magnetic metal nanoparticles for use as more sensitive reagents in magnetic immunoassays.

In theory, any material can be made amorphous if it is cooled fast enough from a melted state, but the required cooling rate for a pure metal has been calculated to be around 10¹⁰ K s⁻¹. Bulk amorphous metals contain at least one or more magnetic transition metals, namely cobalt, iron, or nickel, as well as one or more glass forming elements like phosphorus, boron, or silicon, to lower the critical cooling rate. In commercial production of bulk amorphous metals, a melt containing roughly 80% transition metals and 20% glass formers is cooled at a rate of 100,000 K s⁻¹ to create a metal glass. Stresses induced during manufacturing, variations in composition, and surface effects can induce compositional anisotropy which results in magnetic anisotropy and coercivity. However, when these stresses are minimized and with certain compositions, amorphous metals can have zero magnetic anisotropy and a magnetic permeability which is 10³-10⁵ times higher than crystalline metals.

### SUMMARY OF THE INVENTION

In one aspect the present provides a method for the production of a colloidal suspension of amorphous magnetic metal nanoparticles which have applications that include, but are not limited to, use as a magnetic tag in magnetic immunoassay methods.

In at least one embodiment, a bulk target composed of a mixture of at least one magnetic transition metal selected from the group of elements consisting of Ni, Co, and Fe together with at least one glass formers selected from the group of elements consisting of Si, P, and B is used in a pulsed laser ablation (PLA) process to generate amorphous magnetic metal nanoparticle colloids. The bulk target material can be either crystalline or amorphous. The target surface is cleaned with hydrofluoric acid (HF) to remove any surface oxide and then it is submerged in an organic, oxygen-free solvent such as toluene, acetonitrile, or chloroform which has been degased and is maintained in an oxygen-free environment. A pulsed laser is focused on the target in a PLA process which produces a colloidal suspension of amorphous magnetic metal nanoparticles in the organic solvent. The particles are then coated with an oxygen impermeable coating material such as Au, SiO₂, or C, to make their surface inert and which also allows for subsequent attachment of biomolecules in any known biofunctionalization process. After coating, the particles are transferred to water and the surface of the inert material is modified with appropriate biomolecules.

The colloidal suspension of amorphous magnetic metal nanoparticles can also be used as a tag in magnetic immunoassay methods. Immunoassays rely on the ability of an antibody to recognize and bind a specific antigen in what might be a complex mixture of macromolecules and mixed antigens. The binding event is detected by a tag which is attached to the antibody or the antigen. In the present invention the tag is the amorphous magnetic metal nanoparticle. A Giant Magnetoresistance (GMR) sensor is one example of a magnetometer which can detect the tiny magnetic moments of the amorphous magnetic metal nanoparticles. Amorphous magnetic metal nanoparticles are paramagnetic at sizes larger than crystalline magnetic nanoparticles, which means they can provide more signal per binding event by incorporating more magnetic material while still maintaining the large magnetic permeability necessary for signal processing in an AC magnetic field.

### BRIEF DESCRIPTION OF THE DRAWINGS

FIG. 1 is a schematic diagram depicting one method for detecting antigen macromolecules in a magnetic immunoassay method according to the present invention;

FIG. 2 is a transmission electron micrograph of amorphous magnetic metal nanoparticles prepared by pulsed laser ablation in acetonitrile according to the present invention;

FIG. 3 is a trace of an energy dispersive x-ray analysis of a sample of the composition of the nanoparticles shown in the transmission electron micrograph from FIG. 2;

FIG. 4 is a select area x-ray diffraction of a sample of the nanoparticles shown in the transmission electron micrograph from FIG. 2;

FIG. 5 is a transmission electron micrograph of an amorphous magnetic metal nanoparticle coated with a 5 nm thick layer of graphene;

FIG. 6 shows magnetic hysteresis curves of the colloidal suspension of amorphous magnetic metal nanoparticles coated with graphene in water from FIG. 5 plus curves from two other commercially available magnetic nanoparticles for comparison purposes;

FIG. 7 shows the AC magnetic susceptibility of the three types of amorphous magnetic metal nanoparticles shown in FIG. 5;

FIG. 8 shows the FT-IR spectra of the amorphous metal nanoparticles from the transmission electron micrograph in FIG. 2; and

FIG. 9 shows the resistance change in parts per million on a magnetic immunoassay sensor of the amorphous metal nanoparticles from the transmission electron micrograph in FIG. 2.

### DETAILED DESCRIPTION

In the present application the following terms are defined as followed unless otherwise indicated.

“Nanoparticles” refers to particles having a size ranging from about 1 nanometer (nm) to 0.5 micrometers (μ) in at least one dimension.

“Colloidal suspension” refers to particles suspended in solution by Brownian motion.

“Superparamagnetic” refers to a ferromagnetic material which is composed of nanoparticles smaller than the normal magnetic domain size such that the energy barrier between the parallel and anti-parallel magnetic orientations is much smaller than in the bulk material.

Femtosecond pulsed laser ablation (PLA) in a liquid offers the possibility of producing a colloidal suspension of nanoparticles of amorphous metal which can then be coated with an inert material to prevent oxidation. A single laser pulse can heat metals beyond their boiling point and experiments and molecular dynamics studies have shown that, under certain conditions, clusters of the bulk target can be ejected which maintain the stoichiometry of the bulk material. A few compound semiconductors have been produced in this manner as shown in Semaltianos, N. G. et al. CdTe nanoparticles synthesized by laser ablation. Applied Physics Letters 95, 033302 (2009); Chubilleau, C., Lenoir, B., Migot, S. & Dauscher, a Laser fragmentation in liquid medium: a new way for the synthesis of PbTe nanoparticles. Journal of colloid and interface science 357, 13-7 (2011); and Lalayan, a. a. Formation of colloidal GaAs and CdS quantum dots by laser ablation in liquid media. Applied Surface Science 248, 209-212 (2005). The same method has been used to produce one compound intermetallic as shown in Hagedorn, K., Liu, B. & Marcinkevicius, A. Intermetallic PtPb Nanoparticles Prepared by Pulsed Laser Ablation in Liquid. Journal of the Electrochemical Society 160, F106-F110 (2012). Furthermore, when the melted nanoparticles are ejected into the liquid above the target surface, the cooling rate is 100,000 to 500,000 K s⁻¹, which is sufficient to freeze melted metal into an amorphous configuration. Use of PLA in a liquid offers considerable freedom in choice of solvent, which makes it easier to coat a thin layer of inert material onto the amorphous metal nanoparticles that are formed, to prevent the material from oxidizing and also to provide a surface for further biofunctionalization of the nanoparticles.

In at least one embodiment a bulk target material is provided as a first step. The bulk target can either by crystalline or amorphous; it does not matter because the process will convert it into amorphous nanoparticles. The bulk target comprises at least one magnetic transition metal selected from the group consisting of nickel, iron, cobalt and mixtures thereof. The bulk target further comprises at least one glass former selected from the group consisting of phosphorous, boron, silicon, and mixtures thereof. Preferably the composite bulk target has a composition of XₐY₍₁₋ₐ₎, wherein X is one or more magnetic transition metals and Y is one or more glass formers. The value of “a” is from 0.45 to 0.9, from 0.6 to 0.9, preferably from 0.6 to 0.85, more preferably from 0.75 to 0.8. The bulk target can be in any shaped form including a cylinder, a puck, a rectangle, or a ribbon. The bulk target material is wiped down with a solution of hydrofluoric acid (HF) just prior to use to remove surface oxides. Other oxide removal solvents or solutions can be used. The PAL process is preferably conducted under a degassed oxygen-free atmosphere of nitrogen in a glove box to prevent oxidation of the formed nanoparticles.

The wiped bulk target is placed in a solvent in an oxygen-free atmosphere, preferably in a glove box. The solvent can be any organic solvent and preferably the solvent has no oxygen in its structure to prevent surface oxidation of the formed nanoparticles, by reactive oxygen released during the ablation process. Suitable solvents include toluene, chloroform, or acetonitrile, or fluorinated solvents. The bulk target in the solvent is then subjected to PAL. The preferred PAL parameters are described below. Preferably the pulse duration is about 1 picosecond or less, more preferably the pulse duration is about 500 femtoseconds or less. The pulse energy is preferably from about 1 to 10 μJoules at a pulse repetition rate of from about 100 kHz to 1000 kHz. The laser can be any suitable source having a wavelength of from about 1100 to 300 nm and preferably at an output of from about 0.4 to 1.06 W. The laser beam is scanned over the surface of the bulk target at a rate of from 5 to 10 m/s and preferably is focused at a level of from about 0 to 300 microns (μm) below the surface of the bulk target. In a preferred embodiment the laser parameters as described above produce a laser fluence of from about 0.5 to 1±0.05 Joules/cm². As described above the laser causes superheating of the bulk target and expulsion of nanoparticles that can be cooled sufficiently rapidly by the solvent to result in an amorphous structure of the nanoparticles. The produced amorphous nanoparticles are collected and the solvent is stabilized by addition of any salt at levels of from about 50 μmol to 5 mmol. One such salt comprises tetraoctylammonium bromide.

The collected nanoparticles are then coated with an inert material to prevent oxidation and to provide functional groups to which biomolecules can be attached. Suitable coating materials include gold, carbon, silicon oxide, which are all inert and have well known methods for conjugating biomolecules to their surface, and any other inert material that can be biofunctionalized. Preferably the coating is from about 3 to 50 nm thick on the outside of the nanoparticles. Coating procedures are well known in the art and are preferably carried out under an oxygen-free environment. Once the coating is completed the coated nanoparticles are isolated from the reaction solvent and brought up in an aqueous solution as a stable colloidal suspension of coated amorphous magnetic metal nanoparticles. The nanoparticles can be purified from the aqueous solution using a magnetic separator as known in the art. A preferred aqueous solution is deionized water.

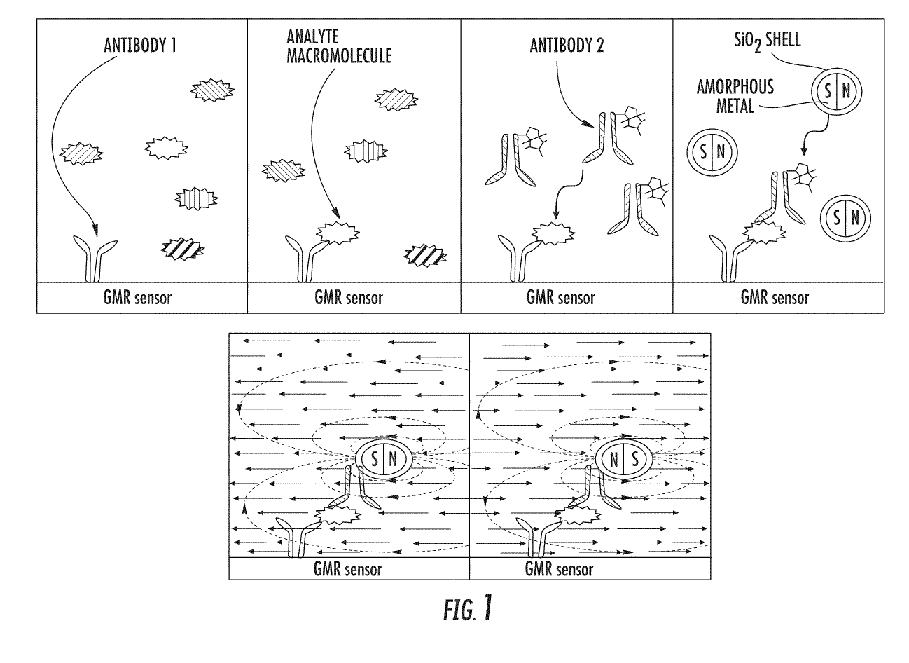

Use of Giant magnetoresistive (GMR) sensors is known as disclosed in the online publication by Gaster, R. S. et al. (2009), Matrix-insensitive protein assays push the limits of biosensors in medicine, Nature medicine, 15 (11) 1327-1332.doi:10.1038/nm.2032. As described in the publication GMR sensors, originally developed for use a read heads in hard-disk drives, are multilayer thin-film structures that operate on the basis of a quantum mechanical effect. A change in the local magnetic field induces a change in the resistance of the sensor which can be measured and quantified. By way of example, FIG. 1 is an adaptation of FIG. 1 d-h of the publication and shows a schematic of one immunoassay method according to the present invention. FIG. 1 shows a matrix-insensitive detection assay in which an array of GMR sensors is used to detect binding events of antigens to arrays of surface-bound antibodies through the use of magnetic nanoparticle tags. In this method a GMR sensor is initially coated with an antibody directed to the antigen macromolecule of interest. The sample solution is then exposed to the GMR sensor and the antigen binds to the antibody bound on the GMR sensor. Then a second antibody that has previously been tagged with the amorphous magnetic metal nanoparticles of the present invention is exposed to the GMR sensor. The second antibody binds to the antigen and because of the magnetic tag this binding is detectable and quantifiable by the GMR sensor system. The signal generated by the nanoparticle binding event is related to the magnetic permeability, which in turn is related to the magnetic moment and magnetic susceptibility, of the magnetic nanoparticles bound on the second antibody. Similarly, the antigen may be bound to the sensor surface and the antibody may have the nanoparticle attached.

By way of example, the following procedure may be used to produce amorphous magnetic metal nanoparticles particles according to the present invention. In this section, all chemicals were used as received. Amorphous magnetic metal nanoparticles were prepared by pulsed laser ablation in acetonitrile of a ribbon of a bulk target material. The bulk target material ribbon comprised 75% by weight Co, 5-10% by weight Fe, 5-10% by weight Ni, 7-15% by weight Si, and 7-15% by weight B for a total weight of 100%. The ribbon was wiped down with HF to remove the surface oxide after the ribbon had been loaded into a nitrogen atmosphere. Oxygen and water were removed from the acetonitrile using molecular sieves and the PLA process was done under a nitrogen atmosphere. An IMRA America D-1K fiber laser system was used to produce the nanoparticles by the PLA process. The laser output was attenuated to 0.75 W and a repetition rate of 500 kHz, 2 μs pulse repetition, was used with a pulse duration of about 600 femtoseconds (fs), yielding pulse energies of 5.2 μJ. A Scanlab HurrySCAN II system was used to scan the laser beam across the amorphous magnetic metal ribbon. The laser was focused 20 μm below the target surface. The fluence with this lens and these laser conditions was estimated to be 0.73±0.05 J cm⁻².

By way of example, the following procedure may be used to coat amorphous magnetic metal nanoparticles, produced as described above, with graphene. The colloidal suspension in acetonitrile was diluted by half with acetone and the laser from the previous section was focused into the solution, with a stir bar circulating the nanoparticles. Decomposition of the solvent by the laser produced a uniform graphene layer 1-10 nm thick, depending on the duration of this step, onto the nanoparticles. The laser output was attenuated to 1.02 W and a repetition rate of 500 kHz, 2 μs pulse repetition, was used with a pulse duration of about 600 fs, yielding pulse energies of 5.2 μJ. The growth rate of the graphene was roughly 1 nm per minute on the nanoparticles.

The graphene can be used to anchor biomolecules to the nanoparticle as the topmost layer of graphene will oxidize to graphitic oxide, which can incorporate carboxylic acid groups. Biomolecules can be conjugated to the carboxylic acid groups by the well-known EDC/Sulfo-NHS reaction. For demonstration purposes in the present invention, Streptavidin was loaded onto the surface of the nanoparticles produced according to the present invention. The sensor surface was loaded with various levels of Biotin, which binds Streptavidin very tightly. In the present demonstration of the use of our process the amorphous magnetic metal nanoparticles were bound with the Streptavidin as described above. When the Streptavidin subsequently binds to the Biotin on the surface of the GMR sensor this binding is detectable, as shown below, by a change in resistance. The difference between the reference sensor pad and the ones having Biotin-Streptavidin bound is measured in parts per million resistance change.

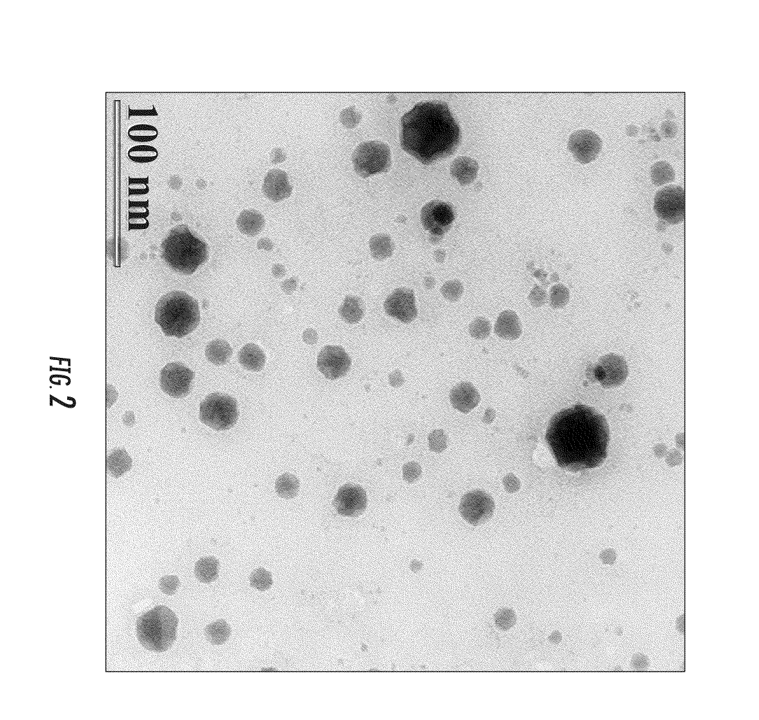

FIG. 2 shows a transmission electron micrograph of amorphous magnetic metal nanoparticles produced by PLA according to the present invention in acetonitrile as described above.

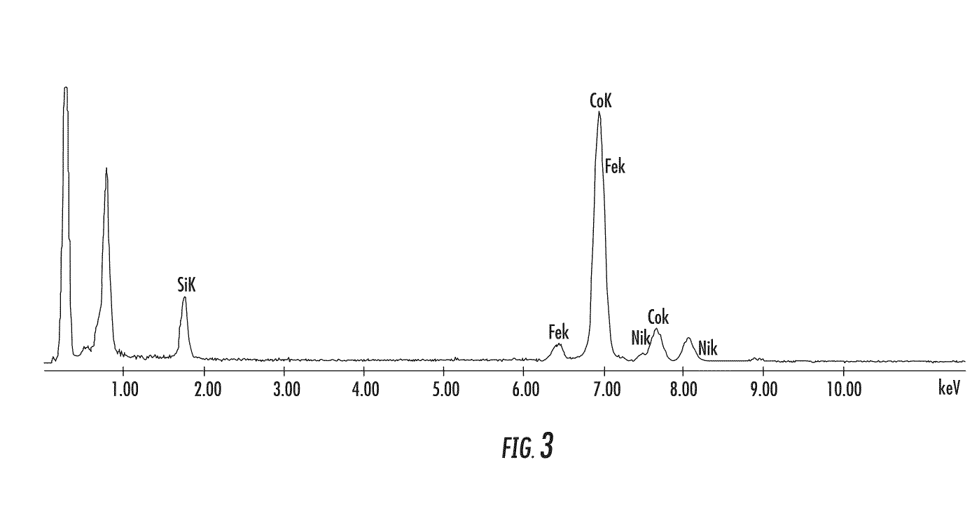

In FIG. 3 a trace of an energy dispersive x-ray analysis of a sample of the composition of FIG. 2 is shown. The results from FIG. 3 show that the nanoparticles prepared according to the present invention have a composition similar to the bulk target they are derived from and that they contain multiple magnetic transition metals and glass formers.

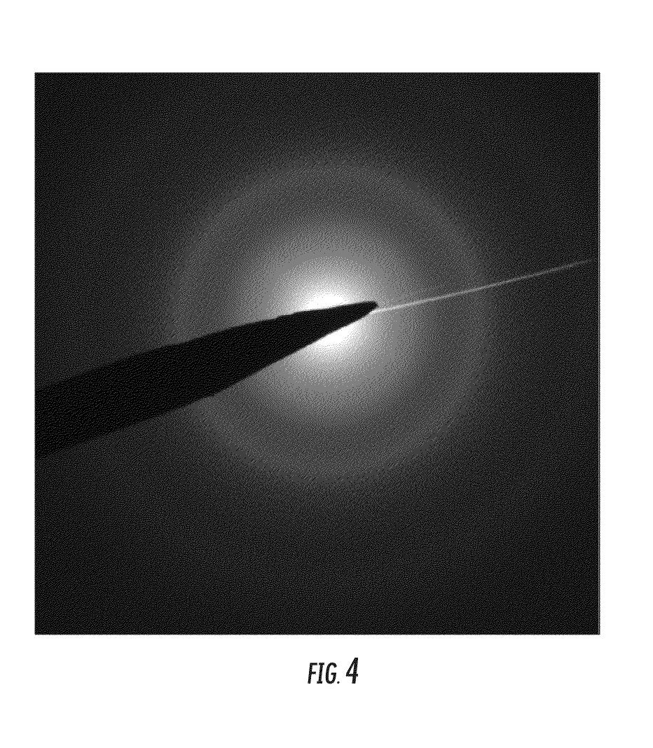

FIG. 4 shows a select area x-ray diffraction of a sample of the composition of nanoparticles from FIG. 2, the halos in the figure confirm the amorphous structure of the nanoparticles. The center indicates the average distance between nearest neighbor atoms and the width indicates the variation in the distance between nearest neighbors.

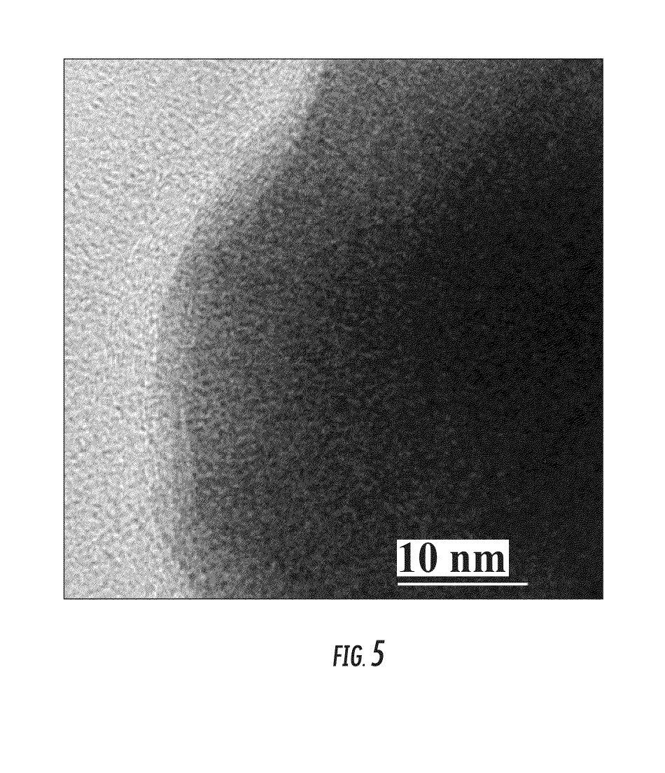

FIG. 5 is a transmission electron micrograph of the amorphous magnetic metal nanoparticles prepared according to the present invention as described above after they were coated with a 5 nm thick layer of graphene using the process described above.

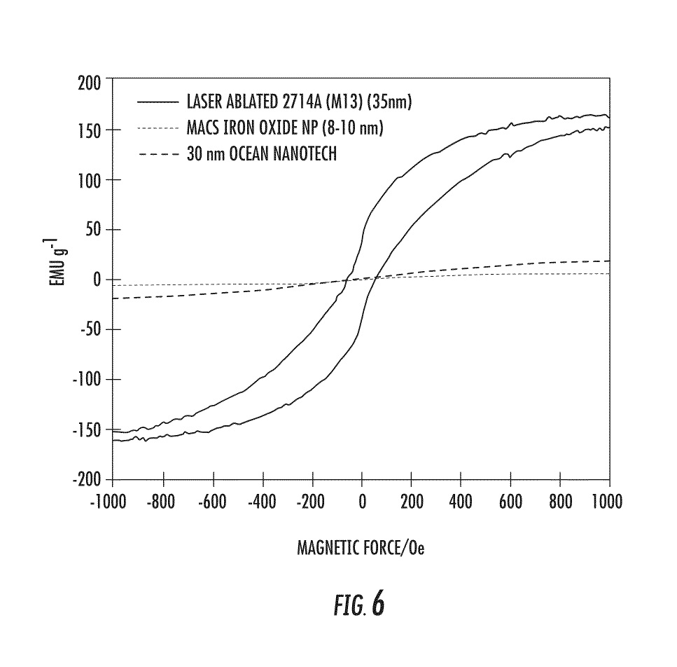

FIG. 6 shows the magnetic hysteresis curves for the following samples: pulsed laser ablated amorphous magnetic metal nanoparticles produced according to the present invention as described above; MACS iron oxide nanoparticles obtained from Miltenyi Biotec; and iron oxide nanoparticles from Ocean Nanotech. A forward and reverse sweep for each sample is shown; however, because of their small size the two sweeps are indistinguishable from each other for the MACS iron oxide and Ocean Nanotech samples. The results demonstrate that the magnetic moment, as measured in emu g⁻¹, of the laser ablated nanoparticles according to the present invention is much larger than that produced by the commercially available iron oxide particles.

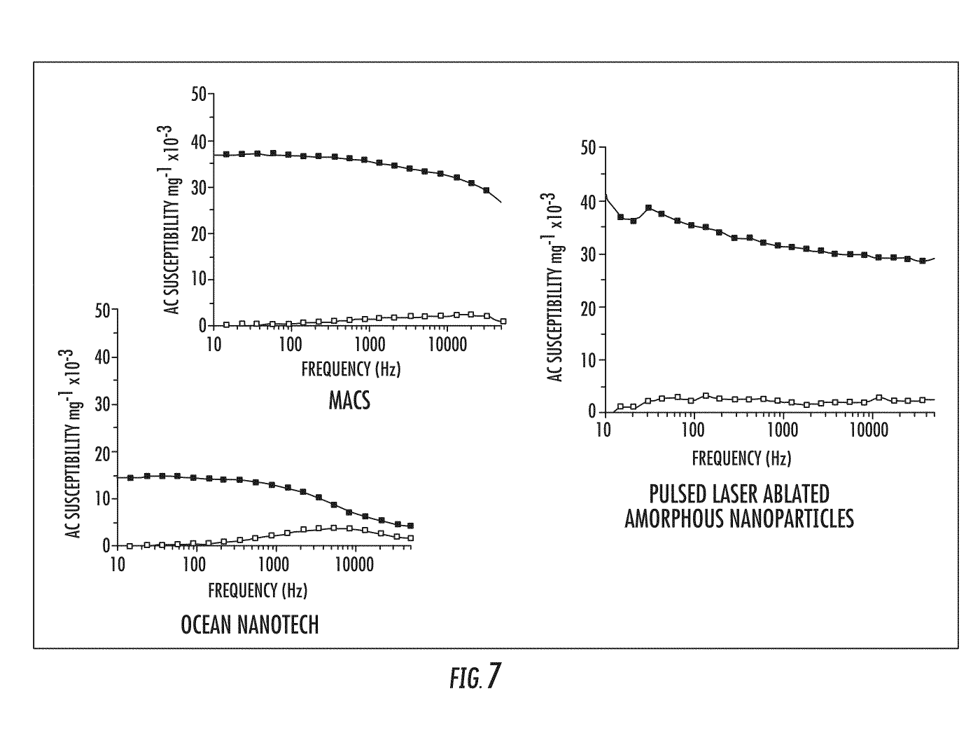

FIG. 7 shows the AC magnetic susceptibility of the three different nanoparticle populations versus frequency. In the first panel the AC susceptibility of the MACS iron oxide particles is shown, these are commercially available. In the second panel the AC susceptibility of commercially available nanoparticles from Ocean Nanotech is shown. Finally, in the third panel the AC susceptibility of the laser ablated nanoparticles produced according to the present invention as described above is shown. The lower trace in each panel is the imaginary component of the susceptibility and the upper trace in each is the real component of the susceptibility. The results demonstrate that the amorphous magnetic metal nanoparticles produced according to the present invention have an AC susceptibility that is comparable to that exhibited by the iron oxide MACS particles.

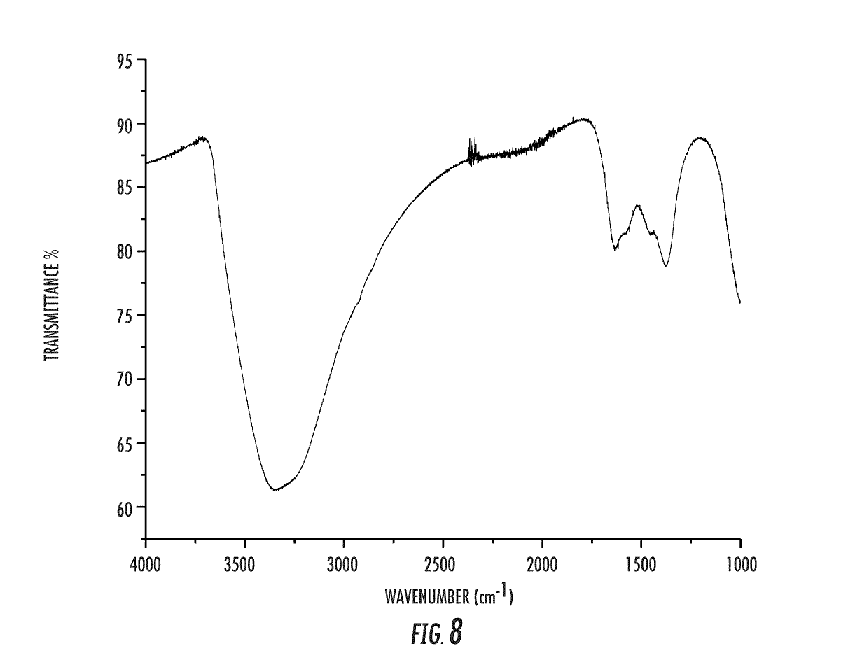

FIG. 8 shows the FT-IR spectra of a sample of the nanoparticles shown in FIG. 2. The characteristic peak shape between 1400 and 1600 nm indicates the presence of carboxylic acid on oxidized graphene, which can be used to anchor proteins and other biomolecules to the nanoparticles.

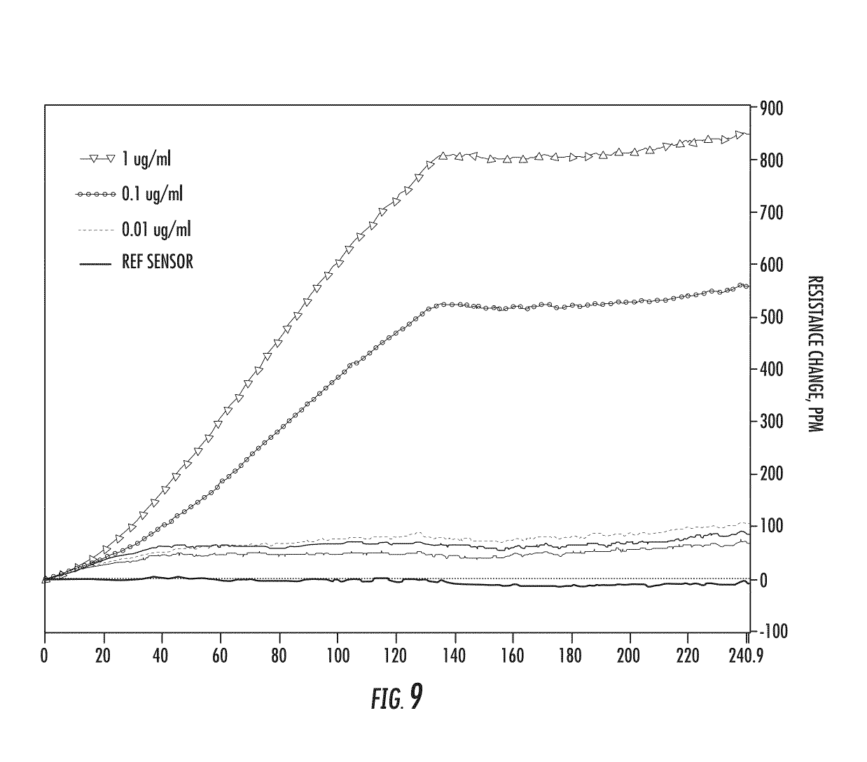

FIG. 9 shows the average signal as a function of Biotin loading on the sensor surface. The load levels were 0.001 μg/ml, 0.01 μg/ml, 0.1 μg/ml and 1.0 μg/ml. The signal was not detectable at 0.001 μg/ml which is labeled as ref sensor in the figure. Significant readings were detectable at levels of 1.0 and 0.1 μg/ml of Biotin.

In at least one embodiment the present invention is a method for fabricating a colloidal suspension consisting of amorphous magnetic metal nanoparticles coated with an anti-oxidation protective layer and suspended in an aqueous solvent, the method comprising the steps of: providing a bulk target of a metal composite having a composition of XₐY₍₁₋ₐ₎, wherein X is at least one magnetic transition metal selected from the group consisting of Fe, Co, Ni, and mixtures thereof and wherein Y is at least one glass former selected from the group consisting of Si, P, B and mixtures thereof; placing the bulk target in a degassed and oxygen-free organic solvent and subjecting the bulk target to pulsed laser ablation, thereby producing a stable colloidal suspension of amorphous magnetic metal nanoparticles in the solvent; coating the nanoparticles in the solvent with an inert coating material capable of preventing oxidation and providing functional groups that can be conjugated to biomolecules; and isolating the coated nanoparticles from the organic solvent into an aqueous solvent.

In at least one embodiment the method comprises providing a bulk target wherein the value of a is from 0.45 to 0.9.

In at least one embodiment the method comprises providing a bulk target wherein the value of a is from 0.6 to 0.85.

In at least one embodiment the method comprises placing the bulk target in an organic solvent which lacks oxygen in its molecular structure.

In at least one embodiment the method comprises placing the bulk target in an organic solvent selected from the group consisting of toluene, chloroform, and acetonitrile.

In at least one embodiment the method uses pulsed laser ablation comprising use of pulses having a pulse duration of less than about 1 picosecond.

In at least one embodiment the method uses pulsed laser ablation comprising use of pulses having a pulse duration of less than about 500 femtoseconds.

In at least one embodiment the method comprises use of a laser wherein the fluence of the pulsed laser ablation is in the range of from about 0.5 to 1 Joules/cm².

In at least one embodiment the method comprises coating the nanoparticles with an inert coating material comprising Au, C, or SiO₂.

In at least one embodiment the method comprises providing a bulk target wherein the value of a is from 0.6 to 0.9.

In at least one embodiment the invention comprises a method for enhancing the sensitivity of a magnetic immunoassay comprising using amorphous magnetic metal nanoparticles produced according to the method as an antibody tag in the magnetic immunoassay.

In at least one embodiment the method for enhancing the sensitivity of a magnetic immunoassay further comprises using a giant magnetoresistance sensor as a magnetometer in the immunoassay.

In at least one embodiment the present invention is a colloidal suspension consisting of: amorphous magnetic metal nanoparticles coated with an anti-oxidation protective inert layer and suspended in an aqueous solvent; the magnetic metal nanoparticles having a composition of XₐY₍₁₋ₐ₎, wherein X is at least one magnetic transition metal selected from the group consisting of Fe, Co, Ni, and mixtures thereof and wherein Y is at least one glass former selected from the group consisting of Si, P, B and mixtures thereof; and the anti-oxidation protective inert layer provides a plurality of functional groups that can be conjugated to biomolecules.

In at least one embodiment the metal nanoparticles of the colloidal suspension have a value of a of from 0.45 to 0.9.

In at least one embodiment the metal nanoparticles of the colloidal suspension have a value of a of from 0.6 to 0.9.

In at least one embodiment the anti-oxidation protective inert layer comprises Au, C, or SiO₂.

In at least one embodiment the anti-oxidation protective inert layer comprises graphene.

In at least one embodiment the functional groups comprise carboxylic acid groups.

In at least one embodiment the anti-oxidation protective inert layer is about 1 to 50 nm thick.

In at least one embodiment the nanoparticles are produced from a bulk material consisting of 75% by weight Co, 5 to 10% by weight Fe, 5 to 10% by weight Ni, 7 to 15% by weight Si, and 7 to 15% by weight B, for a total of 100% by weight all based on the total weight of the bulk material.

The foregoing invention has been described in accordance with the relevant legal standards, thus the description is exemplary rather than limiting in nature. Variations and modifications to the disclosed embodiment may become apparent to those skilled in the art and do come within the scope of the invention. Accordingly, the scope of legal protection afforded this invention can only be determined by studying the following claims.

What we claim is:

1. A method for fabricating a colloidal suspension consisting of amorphous magnetic metal nanoparticles coated with an anti-oxidation protective layer and suspended in an aqueous solvent, the method comprising the steps of:
a) providing a bulk target of a metal composite having a composition of XₐY₍₁₋ₐ₎, wherein X is at least one magnetic transition metal selected from the group consisting of Fe, Co, Ni, and mixtures thereof and wherein Y is at least one glass former selected from the group consisting of Si, P, B and mixtures thereof;
b) placing the bulk target in a degassed and oxygen-free organic solvent and subjecting the bulk target to pulsed laser ablation, thereby producing a stable colloidal suspension of amorphous magnetic metal nanoparticles in the solvent;
c) coating the nanoparticles in the solvent with an inert coating material capable of preventing oxidation and providing functional groups that can be conjugated to biomolecules; and
d) isolating the coated nanoparticles from the organic solvent into an aqueous solvent.

2. The method of claim 1, comprising providing a bulk target wherein the value of a is from 0.45 to 0.9.

3. The method of claim 1, comprising providing a bulk target wherein the value of a is from 0.6 to 0.85

4. The method of claim 1, wherein step b) comprises placing the bulk target in an organic solvent which lacks oxygen in its molecular structure.

5. The method of claim 1, wherein step b) comprises placing the bulk target in an organic solvent selected from the group consisting of toluene, chloroform, and acetonitrile.

6. The method of claim 1, wherein the pulsed laser ablation of step b) comprises use of pulses having a pulse duration of less than about 1 picosecond.

7. The method of claim 1, wherein the pulsed laser ablation of step b) comprises use of pulses having a pulse duration of less than about 500 femtoseconds.

8. The method of claim 1, wherein in step b) the fluence of the pulsed laser ablation is in the range of from about 0.5 to 1 Joules/cm².

9. The method of claim 1, wherein step c) comprises coating with an inert coating material comprising Au, C, or SiO₂.

10. The method of claim 1, comprising providing a bulk target wherein the value of a is from 0.6 to 0.9.

11. A method for enhancing the sensitivity of a magnetic immunoassay comprising using amorphous magnetic metal nanoparticles produced according to the method of claim 9 as an antibody tag in the magnetic immunoassay.

12. The method according to claim 11 further comprising using a giant magnetoresistance sensor as a magnetometer in the immunoassay.

13. A colloidal suspension consisting of:
amorphous magnetic metal nanoparticles coated with an anti-oxidation protective inert layer and suspended in an aqueous solvent;
said magnetic metal nanoparticles having a composition of XₐY₍₁₋ₐ₎, wherein X is at least one magnetic transition metal selected from the group consisting of Fe, Co, Ni, and mixtures thereof and wherein Y is at least one glass former selected from the group consisting of Si, P, B and mixtures thereof; and
said anti-oxidation protective inert layer providing a plurality of functional groups that can be conjugated to biomolecules.

14. A colloidal suspension as recited in claim 13 wherein the value of a is from 0.45 to 0.9.

15. A colloidal suspension as recited in claim 13 wherein the value of a is from 0.6 to 0.9.

16. A colloidal suspension as recited in claim 13 wherein said anti-oxidation protective inert layer comprises Au, C, or SiO₂.

17. A colloidal suspension as recited in claim 16 wherein said anti-oxidation protective inert layer comprises graphene.

18. A colloidal suspension as recited in claim 13, wherein said functional groups comprise carboxylic acid groups.

19. A colloidal suspension as recited in claim 13, wherein said anti-oxidation protective inert layer is about 1 to 50 nm thick.

20. A colloidal suspension as recited in claim 13, wherein said nanoparticles are produced from a bulk material consisting of 75% by weight Co, 5 to 10% by weight Fe, 5 to 10% by weight Ni, 7 to 15% by weight Si, and 7 to 15% by weight B, for a total of 100% by weight all based on the total weight of the bulk material.
